(19) United States
(12) Patent Application Publication
(10) Pub. No.: US 2019/0159854 A1
(43) Pub. Date: May 30, 2019

(54) SURGICAL SYSTEM

(71) Applicant: MEDICAROID CORPORATION, Kobe-shi (JP)

(72) Inventor: Tetsushi ITO, Kobe-shi (JP)

(73) Assignee: MEDICAROID CORPORATION, Kobe-shi (JP)

(21) Appl. No.: 16/262,911

(22) Filed: Jan. 31, 2019

Related U.S. Application Data
(63) Continuation of application No. PCT/JP2018/011613, filed on Mar. 23, 2018.

(30) Foreign Application Priority Data
Mar. 24, 2017 (JP) 2017-059999

(51) Int. Cl. A61B 34/00; A61B 34/30; B25J 9/16

(52) U.S. Cl. CPC A61B 34/71; A61B 34/30; B25J 17/02; A61B 2034/301; B25J 9/1612

(57) ABSTRACT

A grip mechanism to grip a medical treatment tool according to one or more embodiments may include: a base to which a part of the medical treatment tool is attachable; a vertical movement mechanism configured to change a height of the base; and an angle change mechanism configured to change an angle of the base in conjunction with the vertical movement mechanism.

### CROSS-REFERENCE TO RELATED APPLICATION

This application is a continuation of international patent application No. PCT/JP2018/011613 filed on Mar. 23, 2018, which claims priority to Japanese Patent Application No. 2017-059999 filed on Mar. 24, 2017, the entire contents of which is incorporated herein by reference.

### TECHNICAL FIELD

One or more embodiments disclosed herein relate to a surgical system including a grip mechanism configured to grip a medical treatment tool.

### BACKGROUND ART

In recent years, robotic surgical systems have been used in the field of endoscopic surgery.

Medical treatment tools used in the robotic surgical system are configured, for example, by engaging an elongate element, such as wire, with an end effector having a jaw or the like. The elongate element is pulled or fed by the activation of an activation mechanism, configured as a gear or the like, in order to activate the end effector.

A positioning mechanism is used to stably hold and secure such a medical treatment tool. For example, Published Japanese Translation of PCT International Application No. 2016-528946 (Patent Document 1) discloses a tool positioner for a medical treatment tool, in which the tool positioner is capable of gripping a medical treatment tool and adjusting the insertion position and orientation of the medical treatment tool.

### SUMMARY

However, devices, such as the device disclosed in Patent Document 1, which have a mechanism capable of adjusting the position and orientation of a medical treatment tool, may often become large in size.

One or more embodiments are therefore intended to provide a compact grip mechanism capable of adjusting the position and orientation of a medical treatment tool.

A grip mechanism to grip a medical treatment tool according to an aspect of one or more embodiments may include: a base to which a part of the medical treatment tool is attachable; a vertical movement mechanism that changes a height of the base; and an angle change mechanism that changes an angle of the base in conjunction with the vertical movement mechanism.

A grip mechanism to grip a medical treatment tool according to another aspect of one or more embodiments may include: a base to which a proximal end portion of the medical treatment tool is attachable; a gripping portion gripping a distal end portion of the medical treatment tool; a vertical movement mechanism configured to change heights of the base and the gripping portion; and an angle change mechanism configured to change angles of the base and the gripping portion simultaneously.

### BRIEF DESCRIPTION OF THE DRAWINGS

FIG. 1 is a diagram illustrating a configuration of a surgical system according to one or more embodiments.

FIG. 2 is a diagram illustrating a perspective view of a configuration of a medical treatment tool according to one or more embodiments.

FIG. 3 is a diagram illustrating a perspective view of a bundling tube in which guide tubes are inserted.

FIG. 4 is a diagram illustrating a cross-sectional perspective view taken along the line IV-IV in FIG. 3.

FIG. 5 is a diagram illustrating a perspective view of a configuration of a guide tube according to one or more embodiments.

FIG. 6 is a diagram illustrating a cross-sectional view of the guide tube, taken along the line VI-VI in FIG. 5.

FIG. 7 is a diagram generally illustrating a surgical instrument according to one or more embodiments.

FIGS. 8A to 8C are diagrams illustrating a configuration of a distal end portion of a surgical instrument, such as is illustrated in FIG. 7.

FIG. 9 is a diagram illustrating a configuration of a wrist portion of an distal end portion, such as is illustrated in FIGS. 8A to 8C.

FIG. 10 is a diagram illustrating a configuration of a grip mechanism according to one or more embodiments.

FIG. 11 is a diagram for explaining movements of the grip mechanism according to one or more embodiments.

FIG. 12 is a diagram illustrating an enlarged perspective view of a detailed configuration of an angle change mechanism in a grip mechanism, such as is illustrated in FIG. 10.

FIG. 13 is a diagram illustrating a side view of a detailed configuration of the angle change mechanism in a grip mechanism, such as is illustrated in FIG. 10.

FIG. 14 is a diagram illustrating a side view of an angle change mechanism in a grip mechanism, such as is illustrated in FIG. 10, for explaining movements of the angle change mechanism.

FIG. 15 is a diagram illustrating a partial plan view of a detailed configuration of a vertical movement mechanism, such as is illustrated in FIG. 10.

FIG. 16 is a diagram for explaining movements of a vertical movement mechanism, such as is illustrated in FIG. 10.

FIG. 17 is a diagram for explaining a detailed configuration of a third arm portion of a vertical movement mechanism, such as is illustrated in FIG. 16.

FIG. 18 is a diagram illustrating a configuration of a variation of the grip mechanism according to one or more embodiments.

### DETAILED DESCRIPTION

Descriptions are provided hereinbelow for embodiments based on the drawings. In the respective drawings referenced herein, the same constituents are designated by the same reference numerals and duplicate explanation concerning the same constituents is omitted. All of the drawings are provided to illustrate the respective examples only.

### Surgical System

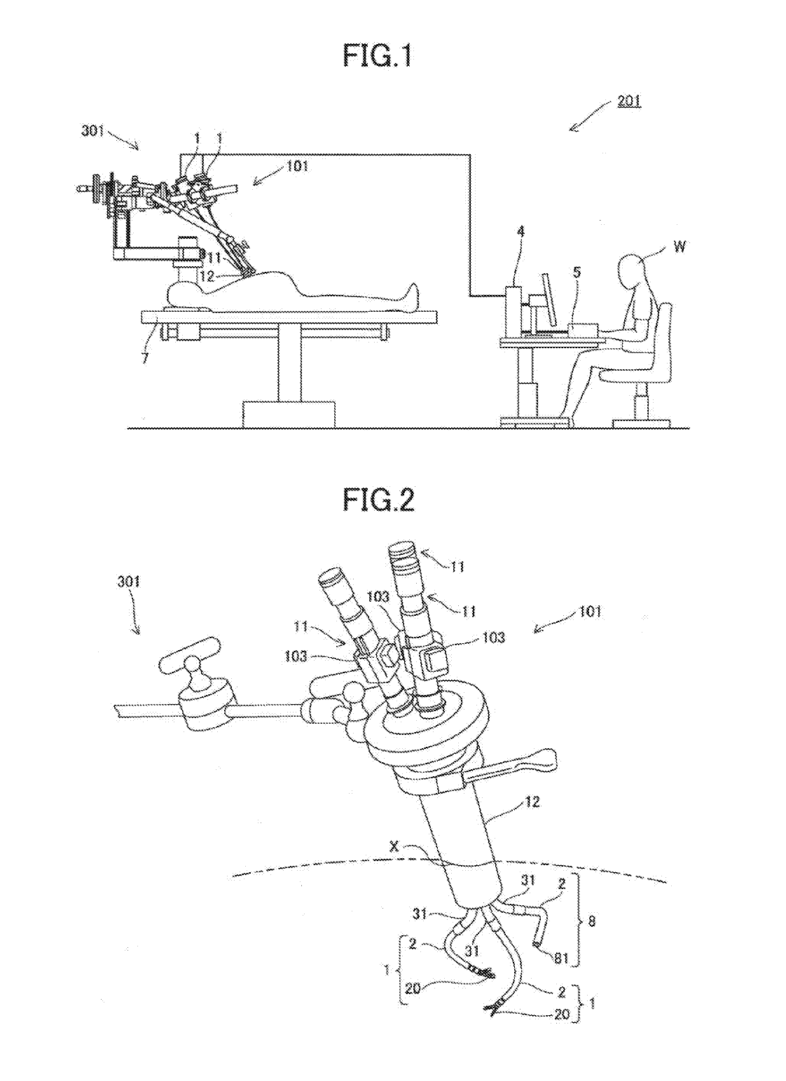

FIG. 1 is a diagram illustrating a configuration of a surgical system according to one or more embodiments. Referring to FIG. 1, a surgical system 201 includes a medical treatment tool 101, a controller 4, a command input unit 5, and a grip mechanism 301. An operator W can perform a surgery, such as an endoscopic surgery, by remotely operating the medical treatment tool 101.

The medical treatment tool 101 includes, for example, one or more surgical instruments 1, one or more endoscopes 8, one or more guide tubes (insertion tubes) 11 in which distal ends of the medical treatment tool 1 and the endoscope 8 are inserted, and a bundling tube (an insertion tube) 12 in which one or more guide tubes 11 are inserted. The surgical instrument 1 and the endoscope 8 are supported by the grip mechanism 301 attached, for example, to a treatment table 7.

The surgical instrument 1, the endoscope 8, the guide tube 11, and the command input unit 5 are electrically connected to the controller 4. The command input unit 5, when operated by an operator W, sends a movement instruction to the surgical instrument 1, the endoscope 8, and the guide tube 11 via the controller 4. The operator W can remotely operate the surgical instrument 1, the endoscope 8, and the guide tube 11 in this manner.

### Medical Treatment Tool

FIG. 2 is a diagram illustrating a perspective view of a configuration of a medical treatment tool according to one or more embodiments. FIG. 2 shows a state in which part of the medical treatment tool 101 is inserted into the body of a patient. In FIG. 2, the medical treatment tool 101 is seen through the body of the patient for illustrative purposes such that the patient's body surface is indicated by a two-dot chain line, and the incision X formed in the patient's body surface is indicated by a solid line.

Referring to FIG. 2, the surgical instrument 1 has an elongate flexible shaft 2 and a distal end portion 20 arranged at a distal end of the flexible shaft 2. In FIG. 2, part of the flexible shaft 2 and the distal end portion 20 pass through the guide tube 11 and are exposed from the guide tube 11.

The endoscope 8 has an elongate flexible shaft 2 and a camera 81 arranged at a distal end of the flexible shaft 2. In FIG. 2, part of the flexible shaft 2 and the camera 81 pass through the guide tube 11 and are exposed from the guide tube 11.

The guide tube 11 is made of soft plastic, such as polypropylene and vinyl chloride. The guide tube 11 has a wire member (not shown) and a guide tube bending adjustment mechanism 103 for operating the wire member.

The guide tube bending adjustment mechanism 103 may be manually controlled to adjust the amount of pulling of the wire member and moreover to stop the movement of the wire member by screwing the wire member, or may be motorized to adjust the amount of pulling of the wire member by means of a gear (not shown) and a motor (not shown) to which the wire member is engaged. The guide tube 11 is bent at a bending portion 31 by adjusting the amount of pulling of the wire member in this manner.

The bundling tube 12 is made of soft plastic, such as polypropylene and vinyl chloride. The bundling tube 12 has a cylindrical shape, the inner diameter of which is larger than the outer diameter of the guide tube 11. The bundling tube 12 has flexibility.

The bundling tube 12, when used in a laparoscopic surgery, is inserted into the body cavity of a patient from the incision X formed in the patient's body surface. Instead of being inserted from the incision X, the bundling tube 12 may be inserted into the patient's body from a natural orifice, such as an oral cavity. That is, the medical treatment tool 101 may be used not only for a laparoscopic surgery, but also for a natural orifice transluminal endoscopic surgery or the like.

The bundling tube 12 is fixed in position and orientation when, for example, the grip mechanism 301 grips the outer wall of a proximal end portion of the bundling tube 12, that is, an end portion not to be inserted through the body surface.

The bundling tube 12, when used, for example, in a laparoscopic surgery, is inserted into the body cavity of a patient from the incision X formed in the patient's body surface. It is therefore difficult to fix the position and orientation of the bundling tube 12, compared to the case in which the bundling tube 12 is inserted in a natural orifice, such as an oral cavity. The grip mechanism 301 configured to grip the bundling tube 12 in the above-described manner is therefore particularly useful in gripping a medical treatment tool used in a laparoscopic surgery.

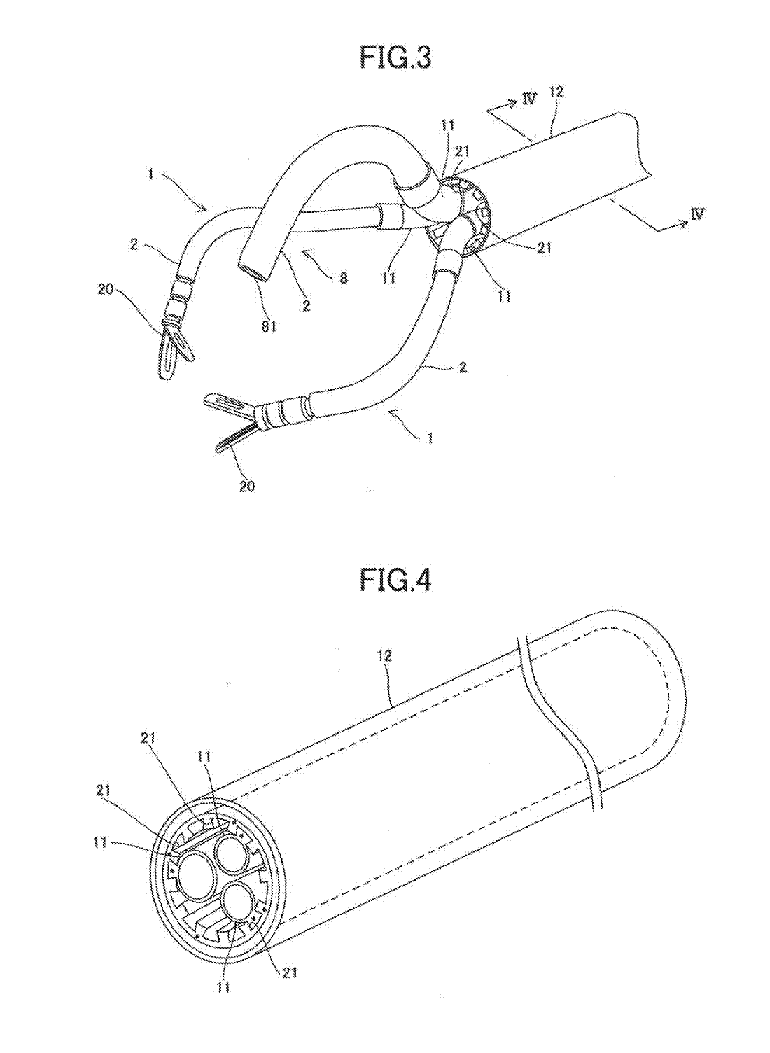

FIG. 3 is a diagram illustrating a perspective view of a bundling tube in which guide tubes are inserted. FIG. 4 is a diagram illustrating a cross-sectional perspective view taken along the line IV-IV of FIG. 3.

Referring to FIGS. 3 and 4, the bundling tube 12 has one or more guide portions 21 which guide the insertion of the guide tube 11. The guide portion 21 is, for example, a dovetail groove formed in the inner wall of the bundling tube 12 and extending in the axial direction of the bundling tube 12. As illustrated in FIG. 4, the guide portion 21 (the guide groove 21) has an approximately trapezoidal cross-section, the width of which gradually increases from the inner circumferential surface to the outer circumferential surface of the bundling tube 12.

As mentioned earlier, the bundling tube 12 has flexibility and can be bent at an appropriate angle for insertion in a body cavity.

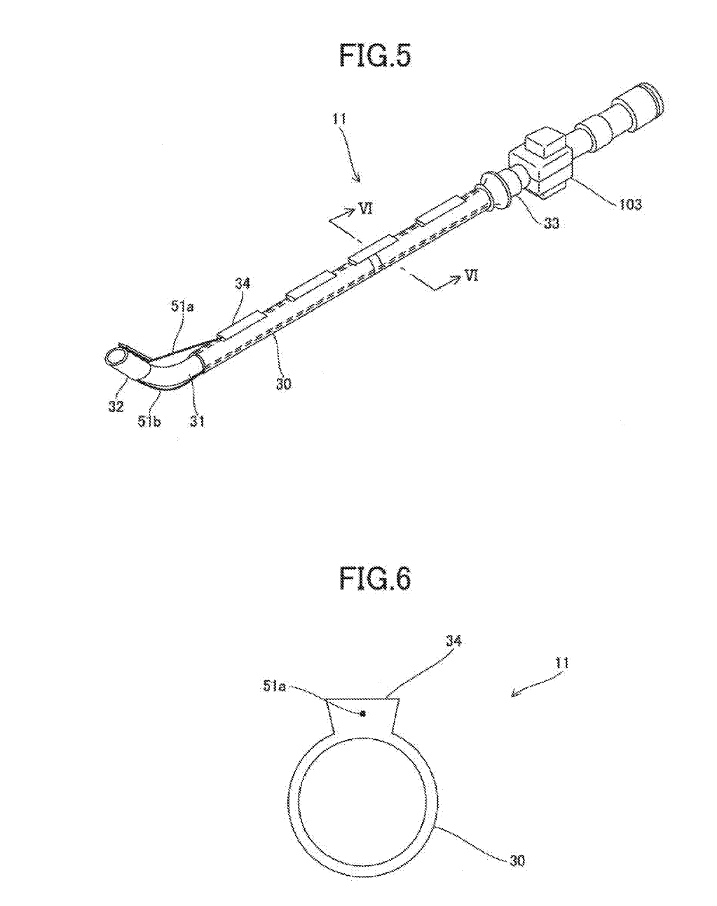

FIG. 5 is a diagram illustrating a perspective view of a configuration of a guide tube according to one or more embodiments.

Referring to FIG. 5, the guide tube 11 includes a flexible shaft portion 30, a bending portion 31, a guide tube distal end portion 32, and a guide tube proximal end portion 33. The guide tube 11 also includes, on the outer circumferential surface of the shaft portion 30, an engaging portion 34 arranged to discontinuously extend in the axial direction of the guide tube 11.

In a state in which the guide tubes 11 are inserted in the bundling tube 12, as illustrated in FIGS. 3 and 4, at least part of the bending portion 31 and the guide tube distal end portion 32 are exposed from the bundling tube 12.

FIG. 6 is a diagram illustrating a cross-sectional view of the guide tube taken along the line VI-VI of FIG. 5.

Referring to FIG. 6, the engaging portion 34 is projected from the outer circumferential surface of the guide tube 11 and has, for example, an approximately trapezoidal cross-section, the width of which gradually increases as the engage portion 34 extends outwardly in the radial direction of the flexible shaft portion 30 of the guide tube 11.

When the guide tubes 11 are inserted in the bundling tube 12, as illustrated in FIGS. 3 and 4, the engaging portion 34 is slidably engaged with the guide portion 21 of the bundling tube 12. This configuration makes it possible to maintain the positional relationship between the bundling tube 12 and the guide tubes 11, inserted in the bundling tube 12, even when the position or the orientation of the medical treatment tool 101 is changed.

The above-described configuration of the engaging portion 34, arranged to discontinuously extend in the axial direction of the guide tube 11, allows the guide tubes 11 to be easily inserted in the bundling tube 12 that is bent. Note that the engaging portion 34 may be configured as a continuous portion extending in the axial direction of the shaft portion 30.

As illustrated in FIG. 5, wire members 51a and 51b are provided as operation elements for operating the guide tube 11. The wire member 51a passes through the inside of the engaging portion 34, and has a first end portion fixed to the guide tube distal end portion 32. The wire member 51b passes through the inside of the shaft portion 30, and has a first end portion fixed to the guide tube distal end portion 32. The guide tube bending adjustment mechanism 103 pulls, or feeds, a second end portion of the wire member 51a or a second end portion of the wire member 51b so as to bend the bending portion 31.

In a case in which it is not necessary to accurately maintain the positional relationship between the bundling tube 12 and the guide tube 11 at the time of adjusting the position or angle of the medical treatment tool 101, the bundling tube 12 does not have to have the above-described guide portion 21, and the guide tube 11 does not have to have the above-described engaging portion 34.

Referring again to FIG. 5, the wire members 51a and 51b, provided as the operation elements for operating the guide tube 11, may be replaced with a plurality of rods, a plurality of flat plates, or a combination of rods and flat plates, which are coupled to one another in a bendable manner.

The operation elements may also be configured as a combination of the wire member 51a and a plurality of rods or a plurality of flat plates. For example, of the operation elements, a portion passing through the engaging portion 34 may be comprised of the wire member 51a, and an exposed portion connecting the engaging portion 34 and the guide tube distal end portion 32 may be comprised of a plurality of rods coupled to one another in a bendable manner.

### Surgical Instrument

### General Configuration

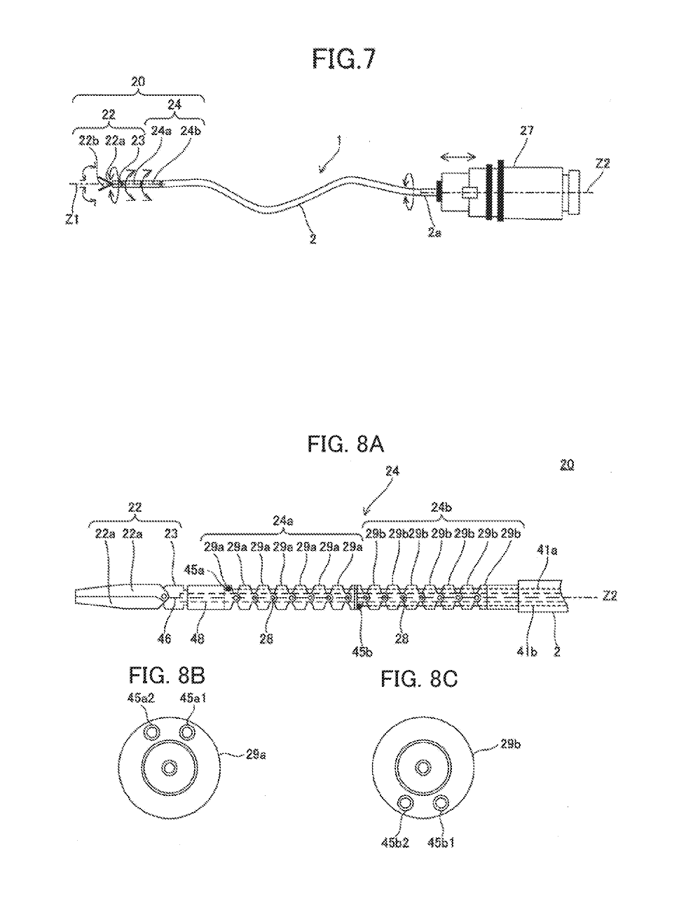

FIG. 7 is a diagram generally illustrating a surgical instrument according to one or more embodiments.

As illustrated in FIG. 7, the surgical instrument 1 has a distal end portion 20, a flexible shaft 2, and a surgical instrument activation mechanism 27. The distal end portion 20 has an end effector 22, such as a grasping forceps, and a multi-articulated portion 24. The end effector 22 has a first jaw 22a, a second jaw 22b, and a wrist portion 23. The multi-articulated portion 24 has a first multi-articulated portion 24a and a second multi-articulated portion 24b.

Note that the end effector 22 is not limited to a grasping forceps, but may also be a scalpel or a hook.

An elongate element, such as wire or cables, which will be described later, is secured to each of the first jaw 22a, the second jaw 22b, the wrist portion 23, the first multi-articulated portion 24a, and the second multi-articulated portion 24b.

The flexible shaft 2 has a proximal end portion 2a opposite to an end toward the distal end portion 20. The proximal end portion 2a is coupled to the surgical instrument activation mechanism 27 in a manner that allows the flexible shaft 2 to rotate.

The wrist portion 23 is in a shape that extends in a particular direction. Specifically, the wrist portion 23 has its first longitudinal end coupled to the first and second jaws 22a and 22b, and its second longitudinal end coupled to the multi-articulated portion 24. The wrist portion 23 is rotatable about a distal end axis Z1 extending in the longitudinal direction of the wrist portion 23.

The surgical instrument activation mechanism 27 includes a plurality of activation pulleys, which will be described later. A plurality of wires, secured to the first jaw 22a, the second jaw 22b, the wrist portion 23, the first multi-articulated portion 24a, and the second multi-articulated portion 24b, are respectively wound around the plurality of activation pulleys. Movements of these wires wound around the activation pulleys activate the first and second jaws 22a and 22b, the wrist portion 23, and the first and second multi-articulated portions 24a and 24b, independently from one another.

The surgical instrument activation mechanism 27 includes, for example, a first external motor (not shown) and a second external motor (not shown). Activation of the first external motor causes the surgical instrument activation mechanism 27 to rotate about a proximal end axis Z2 extending in the longitudinal direction of the proximal end portion 2a. Activation of the second external motor causes the surgical instrument activation mechanism 27 to move along the proximal end axis Z2.

That is, the surgical instrument 1 according to one or more embodiments is configured to be operable in, for example, seven degrees of freedom indicated by the arrows shown in FIG. 7. The surgical instrument 1 may also be configured to be operable in 3 to 6 degrees of freedom with, for example, the first and second multi-articulated portions 24a and 24b being integrally formed.

### Configuration of Distal End Portion

### Multi-Articulated Portion

FIGS. 8A to 8C are diagrams each illustrating a configuration of a distal end portion of a surgical instrument, such as is illustrated in FIG. 7. FIG. 8A is a diagram illustrating a detailed configuration of the multi-articulated portion of the distal end portion. FIG. 8B is a diagram illustrating a state in which a multi-articulated portion operating wire, such as is illustrated in FIG. 8A, is secured to the first multi-articulated portion. FIG. 8C is a diagram illustrating a state in which a multi-articulated portion operating wire, such as is illustrated in FIG. 8A, is secured to the second multi-articulated portion.

As illustrated in FIG. 8A, the first and second multi-articulated portions 24a and 24b of the distal end portion 20 have a plurality of parts 29a and 29b, respectively, which are aligned along the distal end axis Z1 via pins 28.

Each of the parts 29a and 29b has a columnar shape extending in the extension direction of the distal end axis Z1. Both ends of the columnar shape of each of the parts 29a and 29b are tapered.

A multi-articulated portion operating wire 41a extending along the distal end axis Z1 passes through the parts 29a and 29b. A multi-articulated portion operating wire 41b extending along the distal end axis Z1 passes through the parts 29b.

As illustrated in FIG. 8B, both ends of the multi-articulated portion operating wire 41a are secured at distal end securing points 45a1 and 45a2 of the first multi-articulated portion 24a. As illustrated in FIG. 8C, both ends of the multi-articulated portion operating wire 41b are secured at distal end securing points 45b1 and 45b2 of the second multi-articulated portion 24b.

Pulling of one end of the multi-articulated portion operating wire 41a by the surgical instrument activation mechanism 27 illustrated in FIG. 7 causes the first multi-articulated portion 24a to bend. Similarly, pulling of one end of the multi-articulated portion operating wire 41b by the surgical instrument activation mechanism 27 causes the second multi-articulated portion 24b to bend. These configurations of the first and second multi-articulated portions 24a and 24b, which can be bent independently from each other, allow the first multi-articulated portion 24 to be bent into a complicated shape, such as an S-shaped curve.

### Wrist Portion

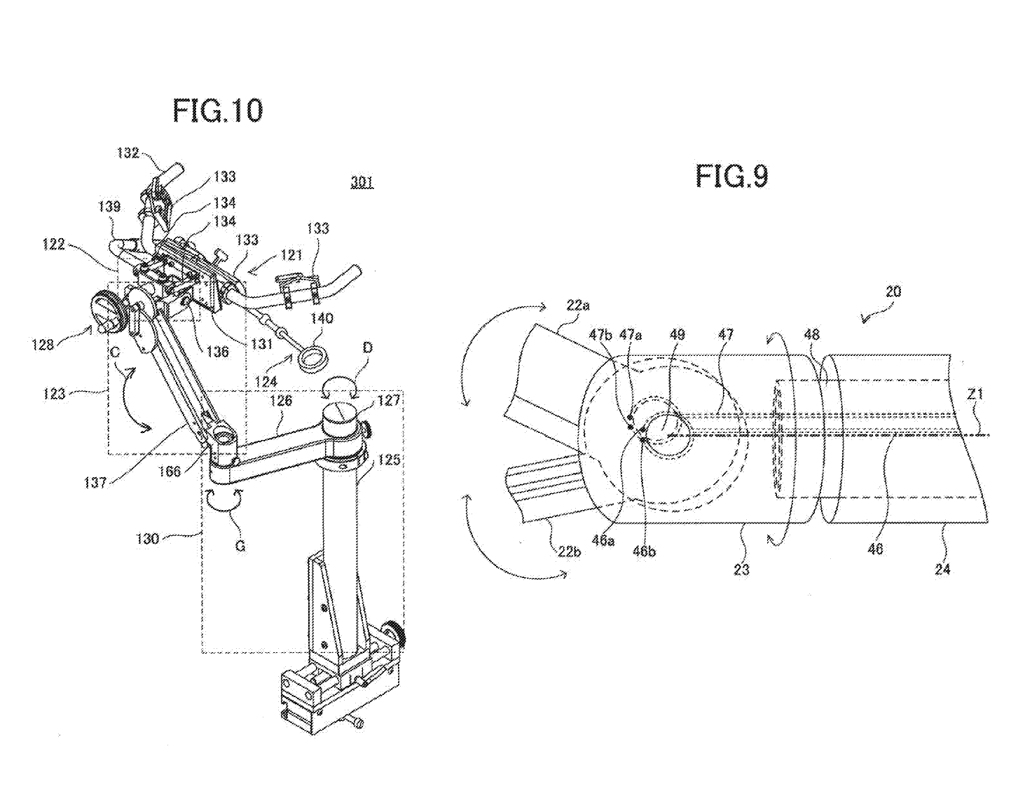

FIG. 9 is a diagram illustrating a configuration of a wrist portion of a distal end portion, such as is illustrated in FIGS. 8A to 8C.

Referring to FIG. 9, a torque transmission tube 48 passes through the interior of the multi-articulated portion 24. More specifically, the torque transmission tube 48 passes through the inside of the multi-articulated portion 24 and the flexible shaft 2, which are illustrated in FIG. 7, with a first end secured to the wrist portion 23 and a second end rotatably coupled to the surgical instrument activation mechanism 27.

The rotation of the torque transmission tube 48 about the proximal end axis Z2, caused by the surgical instrument activation mechanism 27, causes the wrist portion 23 secured to the torque transmission tube 48, and the first and second jaws 22a and 22b coupled to the wrist portion 23, to rotate about the distal end axis Z1.

Note that the wrist portion 23 may be rotated by a wire instead of the torque transmission tube 48. In this case, the mechanism for rotating the wrist portion 23 is configured as described in, for example, WO 2017/006374 (Patent Document 2).

That is, a groove (not shown) is formed in the wrist portion 23 in the circumferential direction about the distal end axis Z1. A first wire and a second wire are used instead of the torque transmission tube 48. The first wire passes through part of the groove, and the second wire passes through another part the groove where the first wire does not pass through.

Pulling of the first or second wire by the surgical instrument activation mechanism 27 causes the wrist portion 23 and the first and second jaws 22a and 22b coupled to the wrist portion 23 to rotate about the distal end axis Z1.

### Jaw

As illustrated in FIG. 9, two jaw operating wires 46 and 47 pass through the interior of the wrist portion 23. The jaw operating wire 46 couples the surgical instrument activation mechanism 27 and the first jaw 22a, illustrated in FIG. 7, to each other. The jaw operating wire 47 couples the surgical instrument activation mechanism 27 and the second jaw 22b, illustrated in FIG. 7, to each other.

More specifically, the jaw operating wire 46 has a first end 46a and a second end 46b which are secured to the first jaw 22a. Pulling of the first end 46a or the second end 46b by the surgical instrument activation mechanism 27 causes the first jaw 22a to rotate about a connecting shaft 49 arranged in the wrist portion 23.

Similarly, the jaw operating wire 47 has a first end 47a and a second end 47b which are secured to the second jaw 22b. Pulling or feeding of the first end 47a or the second end 47b along the proximal end axis Z2 by the surgical instrument activation mechanism 27 causes the second jaw 22b to rotate about the connecting shaft 49.

### Grip Mechanism

### General Configuration

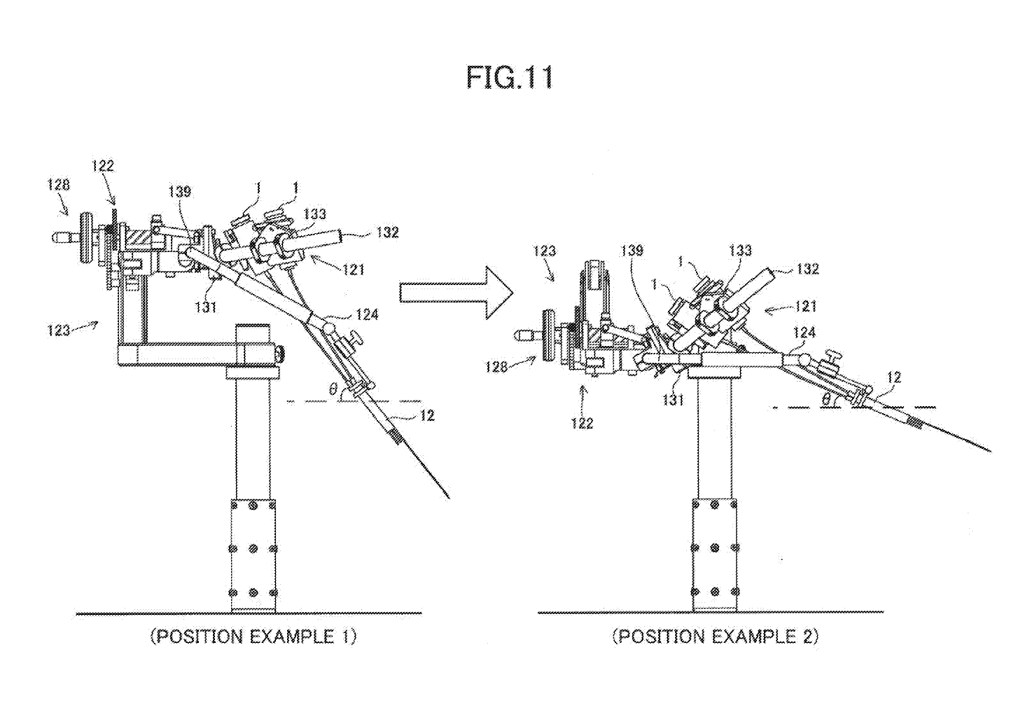

FIG. 10 is a diagram illustrating a configuration of a grip mechanism according to one or more embodiments. FIG. 11 is a diagram for explaining movements of the grip mechanism according to one or more embodiments.

Referring to FIGS. 10 and 11, the grip mechanism 301 includes a base 121, an angle change mechanism 122, a vertical movement mechanism 123, a gripping portion 124, an operating portion 128, and a horizontal position adjusting mechanism 130.

The base 121 has a first support portion 131 coupled to the angle change mechanism 122, a frame 132, and one or more attachment portions 133. The frame 132 is a bar-shaped member attached to the first support portion 131, and has, for example, a circumferentially extending shape, part of which is open.

Specifically, the frame 132 has a substantially U-shape that is bent at a plurality of positions. The shape of the frame 132 may be an arc or the like.

The attachment portion 133 is disposed at any position of the frame 132. The surgical instrument 1 illustrated in FIG. 2 is attachable to the attachment portion 133. The attachment portion 133 is attached to the frame 132 such that the angle of the attachment portion 133 with respect to the frame 132 is adjustable.

This configuration of the frame 132, part of which is open, may facilitate the attachment of the surgical instrument 1 to the attachment portion 133, and also may downsize the grip mechanism 301, compared to a case in which the frame 132 has a closed shape.

The gripping portion 124 has a second support portion 139 coupled to the angle change mechanism 122, and a receiving portion 140. The receiving portion 140 has, for example, a ring shape, and is capable of receiving, for attachment, the bundling tube 12 illustrated in FIG. 2.

The angle change mechanism 122 can change the angles of the base 121 and the gripping portion 124. The vertical movement mechanism 123 can change the height of the base 121 and the height of the gripping portion 124, in conjunction with the angle change mechanism 122.

Specifically, as illustrated in “POSITION EXAMPLE 1” and “POSITION EXAMPLE 2” in FIG. 11, the angle change mechanism 122 changes the angle of the base 121 so that the relative height of the first support portion 131 is changed. That is, the angle change mechanism 122 changes the angle of the base 121 so that an angle θ formed between the longitudinal direction of the surgical instrument 1 attached to the base 121 and the horizontal plane is changed.

Similarly, the angle change mechanism 122 changes the angle of the gripping portion 124 so that the relative height of the second support portion 139 is changed. That is, the angle change mechanism 122 changes the angle of the gripping portion 124 so that an angle θ formed between the longitudinal direction of the bundling tube 12 attached to the gripping portion 124 and the horizontal plane is changed.

Since the bundling tube 12 is flexible and bendable, the angle θ is determined by the posture of the bundling tube 12 defined by the receiving portion 140. The insertion orientation of the medical treatment tool 101, including the bundling tube 12, into the body cavity is roughly determined in this manner. The bundling tube 12 extends linearly when it takes a standard posture. Thus, in the standard posture, the insertion angle of the distal end of the bundling tube 12 into the body cavity substantially coincides with the angle θ formed between the longitudinal direction of the bundling tube 12 and the horizontal plane.

In this manner, the access height and the access angle to the patient, that is, the position and the insertion orientation of the medical treatment tool 101, can be adjusted while maintaining the posture of the entire medical treatment tool 101, from the surgical instrument activation mechanism 27 to the distal end portion 20.

The vertical movement mechanism 123 changes the height of each of the base 121, the angle change mechanism 122, and the gripping portion 124, such that the positions of the base 121, the angle change mechanism 122, and the gripping portion 124 are lowered as the angle θ becomes smaller.

This configuration allows the grip mechanism 301 to grip the surgical instrument 1 and the bundling tube 12 at an appropriate height according to the insertion angle of the surgical instrument 1 with respect to the patient's body surface.

The angle change mechanism 122 and the vertical movement mechanism 123 convert a force applied by the operating portion 128 to a force that changes the angles of the base 121 and the gripping portion 124, and a force that changes the heights of the base 121 and the gripping portion 124, respectively. That is, the angle change mechanism 122 and the vertical movement mechanism 123 utilize a force applied to the same portion.

This configuration may facilitate the operation for changing the angle and height of the medical treatment tool 101 gripped by the grip mechanism 301, and also may simplify the structure of the grip mechanism 301 and hence may downsize the grip mechanism 301.

Detailed configurations of the angle change mechanism 122, the horizontal position adjusting mechanism 130, and the vertical movement mechanism 123 will be described below.

### Detailed Configuration of Angle Change Mechanism

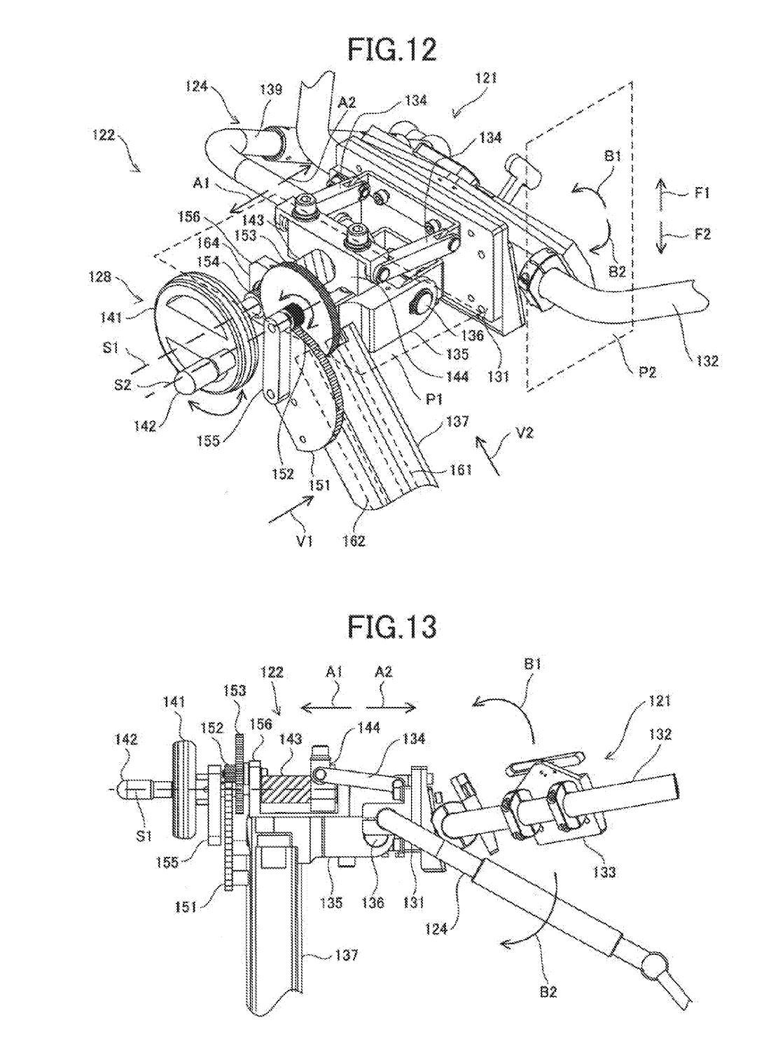

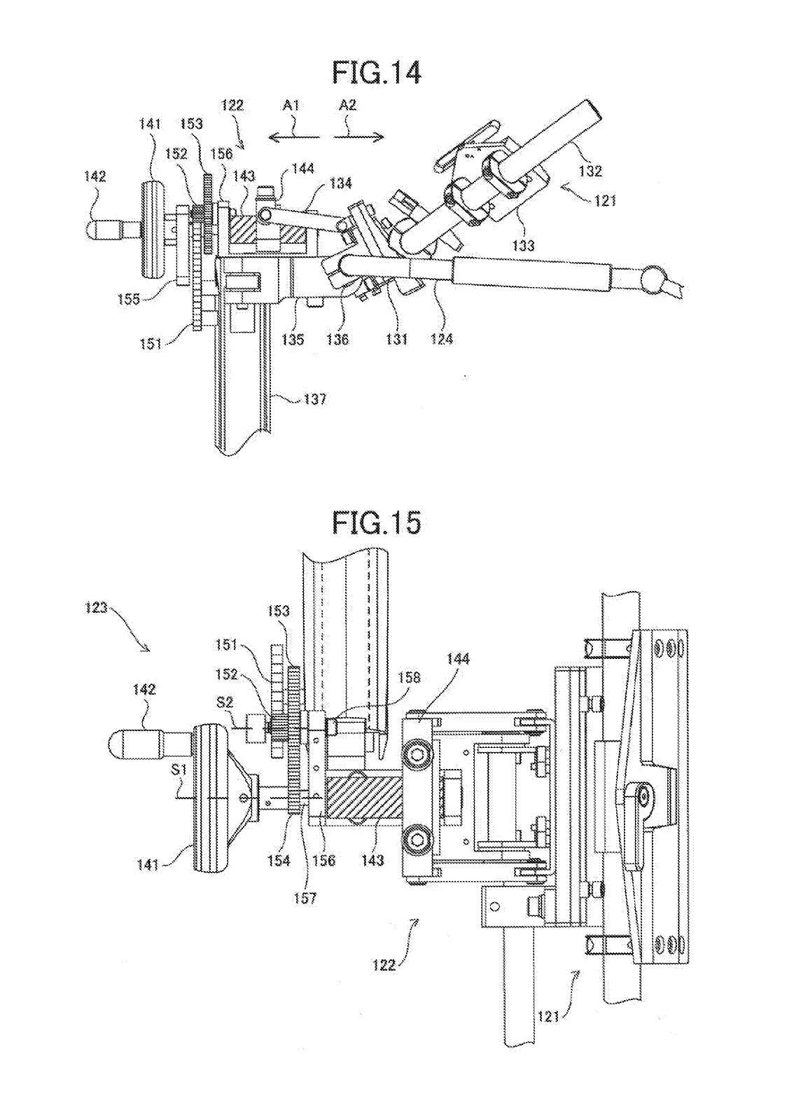

FIG. 12 is a diagram illustrating an enlarged perspective view of a detailed configuration of an angle change mechanism in a grip mechanism, such as is illustrated in FIG. 10. FIG. 13 is a diagram illustrating a side view of a detailed configuration of an angle change mechanism in a grip mechanism, such as is illustrated in FIG. 10. FIG. 14 is a diagram illustrating a side view of an angle change mechanism in a grip mechanism, such as is illustrated in FIG. 10, for explaining movements of the angle change mechanism.

Referring to FIGS. 12 to 14, the angle change mechanism 122 includes: two first coupling portions 134, each having one end connected to the first support portion 131 of the base 121; a first arm portion 135; and a second coupling portion 136 which rotatably couples the first support portion 131 to the first arm portion 135. The two first coupling portions 134 and the second coupling portion 136 extend substantially parallel to the horizontal plane.

The other ends of the two first coupling portions 134 are coupled to a nut portion 144. The extending direction of the two first coupling portions 134 is parallel to the extending direction of a ball screw 143. The extending direction of the two first coupling portions 134 intersects with a rotational axis of the second coupling portion 136 at a 90-degree angle.

The first coupling portions 134 are movable in the direction of the arrow A1 or A2, that is, along the longitudinal direction of the first coupling portions 134. Movements of the first coupling portions 134 cause the first support portion 131 to rotate about the second coupling portion 136.

This rotation causes the gripping portion 124 and the frame 132, which are coupled to the first support portion 131, and the attachment portion 133, as well, to rotate about the second coupling portion 136 in the direction of the arrow B1 or B2. In a state in which the surgical instrument 1 is attached to the attachment portion 133, the angle of the surgical instrument 1 with respect to the horizontal plane is changed by this rotation of the attachment portion 133.

More specifically, the operating portion 128 has a rotating portion 141 and a handle 142. The rotating portion 141 is rotatable about a first axis S1 substantially parallel to the horizontal plane. The handle 142 is coupled to the rotating portion 141. An operator, for example, can rotate the rotating portion 141 by operating the handle 142.

The angle change mechanism 122 further includes the ball screw 143 and the nut portion 144. The first coupling portions 134 are coupled to the nut portion 144.

The ball screw 143 is coupled to the rotating portion 141 and converts the rotational motion of the rotating portion 141 into linear motion of the nut portion 144. Specifically, the nut portion 144 has a groove in the inner peripheral surface of its hole. The groove engages with a groove formed in the outer periphery of the ball screw 143. Rotation of the rotating portion 141 causes the ball screw 143 to rotate about the first axis S1, and causes the nut portion 144 engaged with the ball screw 143 to move linearly in the direction of the arrow A1 or A2.

For example, suppose that the nut portion 144 moves linearly in the direction of the arrow A1 in the state illustrated in FIG. 13. In this case, the nut portion 144 and the first coupling portions 134 move toward the rotating portion 141. The base 121 and the gripping portion 124 coupled to the first coupling portions 134 rotate upward in the direction of the arrow B1 about the second coupling portion 136, to be in the state as illustrated in FIG. 14.

Similarly, in a case in which the nut portion 144 moves linearly in the direction of the arrow A2 in the state illustrated in FIG. 13, the nut portion 144 and the first coupling portions 134 move away from the rotating portion 141. The base 121 and the gripping portion 124 coupled to the first coupling portions 134 rotate downward in the direction of the arrow B2 about the second coupling portion 136.

### Detailed Configurations of Horizontal Position Adjusting Mechanism and Vertical Movement Mechanism

Referring again to FIG. 10, the horizontal position adjusting mechanism 130 includes a support column 125, a second arm portion 126, and a first fulcrum portion 127.

The vertical movement mechanism 123 includes a third arm portion 137 and a coupling base 166. The coupling base 166 rotatably couples the third arm portion 137 to the second arm portion 126. The third arm portion 137 has a first end to which the base 121 and the gripping portion 124 are coupled via the angle change mechanism 122, and a second end coupled to the coupling base 166.

The heights of the base 121 and the gripping portion 124 connected to the third arm portion 137 are changed by rotation of the third arm portion 137 about the coupling base 166.

The first fulcrum portion 127 rotatably couples the second arm portion 126 to the support column 125. The second arm portion 126 has a first end coupled to the third arm portion 137 via the coupling base 166, and a second end coupled to the support column 125 via the first fulcrum portion 127.

Rotation of the second arm portion 126 in a direction of the arrow D on the horizontal plane, that is, rotation of the second arm portion 126 about the first fulcrum portion 127, causes rotations, in the direction of arrow D, of the vertical movement mechanism 123 coupled to the second arm portion 126, the angle change mechanism 122 coupled to the vertical movement mechanism 123, and the base 121 and the gripping portion 124 coupled to the angle change mechanism 122.

The third arm portion 137 rotates, with respect to the second arm portion 126, in a direction of the arrow G along the horizontal plane. The positions of the base 121 and the gripping portion 124 in the horizontal direction are adjusted by a combination of the rotations in the direction of the arrows D and G.

Referring again to FIG. 12, the vertical movement mechanism 123 further includes a first gear 151, a second gear 152, a third gear 153, a fourth gear 154, a gear coupling portion 155, and a fixing portion 156. Rotational axes of the first, second, third, and fourth gears 151, 152, 153, and 154 are all parallel to the first axis S1.

The fourth gear 154 is coupled to the rotating portion 141. The rotation of the rotating portion 141 rotated, for example, by an operator through the handle 142 causes the fourth gear 154 to rotate about the first axis S1.

The third gear 153 engages with the fourth gear 154, and rotates about a second axis S2 parallel to the first axis S1 as the fourth gear 154 rotates. The second gear 152 is coupled to substantially the center of the main surface of the third gear 153, and rotates about the second axis S2 as the third gear 153 rotates.

That is, the rotating portion 141 rotated by an operator through the handle 142 causes the second, third, and fourth gears 152, 153, and 154 to rotate.

The first gear 151 engages with the second gear 152, and is fixed to the third arm portion 137. The gear coupling portion 155 couples substantially the center of the main surface of the second gear 152 and substantially the center of the main surface of the first gear 151 to each other.

FIG. 15 is a diagram illustrating a partial plan view of a detailed configuration of a vertical movement mechanism, such as is illustrated in FIG. 10.

Referring to FIG. 15, the ball screw 143 is coupled to the fixing portion 156. The fourth gear 154 is coupled to the fixing portion 156 via a first shaft 157 extending along the first axis S1. The second and third gears 152 and 153 are also coupled to the fixing portion 156 via a second shaft 158 extending along the second axis S2.

Referring again to FIG. 13, the third arm portion 137 is coupled to the fixing portion 156 via the first arm portion 135.

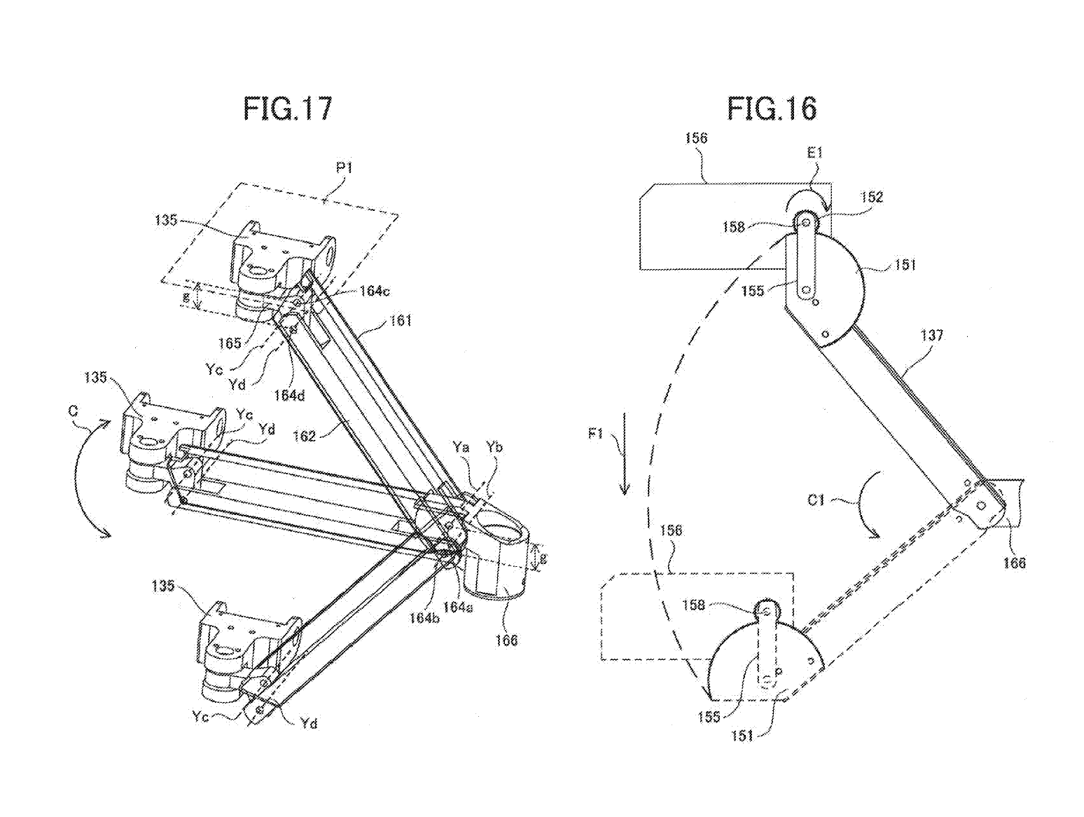

FIG. 16 is a diagram for explaining movements of a vertical movement mechanism, such as is illustrated in FIG. 10. A partial configuration of the vertical movement mechanism 123 is illustrated in FIG. 16.

Referring to FIG. 16, suppose that the second gear 152 is rotated in a direction of the arrow E1 by an operator through the handle 142, while the first and second gears 151 and 152 and the third arm portion 137 are in the position indicated by the solid line shown in FIG. 16.

The second gear 152 is coupled to the fixing portion 156, as mentioned above. The second gear 152 therefore rotates without changing its position in the horizontal direction. The third gear 153 engaged with the second gear 152, and the third arm portion 137 to which the third gear 153 is fixed, therefore rotate on the coupling base 166 in the direction of the arrow C1. As a result, the fixing portion 156 moves in the direction of the arrow F1 along the vertical direction.

The movement of the fixing portion 156 in the direction of the arrow F1 causes the angle change mechanism 122, the base 121, and the gripping portion 124, which are illustrated in FIG. 12, to move in the direction of the arrow F1.

FIG. 17 is a diagram for explaining a detailed configuration of a third arm portion of a vertical movement mechanism, such as is illustrated in FIG. 16. An internal configuration of the third arm portion 137 is illustrated in FIG. 17.

Referring to FIG. 17, the vertical movement mechanism 123 (not shown) further includes: two parallel link members 161 and 162 extending along the longitudinal direction of the third arm portion 137 and parallel to each other; joint members 164a, 164b, 164c, and 164d; and an additional link (a link coupling member) 165 coupling the two parallel link members 161 and 162. The parallel link members 161 and 162 have the same longitudinal length.

The joint members 164a, 164b, 164c, and 164d extend in the direction of axes Ya, Yb, Yc, and Yd, respectively. The axes Ya, Yb, Yc, and Yd are parallel to one another.

The parallel link members 161 and 162 are coupled to the coupling base 166, rotatably coupled to the second arm portion 126 illustrated in FIG. 10, so as to be rotatable about the axes Ya and Yb, respectively. The axes Ya and Yb are separated from each other by a distance g in the vertical direction.

The additional link 165 is provided at end portions of the parallel link members 161 and 162 opposite to the end portions where the coupling base 166 is coupled. More specifically, the additional link 165 is coupled to the parallel link member 161 so as to be rotatable about the upper rotational axis Yc, and is coupled to the parallel link member 162 so as to be rotatable about the lower rotational axis Yd. The upper and lower rotational axes Yc and Yd are separated from each other by a distance g in the vertical direction. The additional link 165 is also connected to the base 121 illustrated in FIG. 10 via the first arm portion 135.

The parallel link members 161 and 162 rotate relative to the coupling base 166 while maintaining the distance g between the axes Ya and Yb and the distance g between the axes Yc and Yd.

In this manner, regardless of the rotation of the parallel link members 161 and 162, the plane including the axis Ya and the axis Yb and the plane including the axis Yc and the axis Yd are kept parallel to each other, thereby maintaining a main surface (hereinafter referred to as a “plane P1”) of the first arm portion 135, coupled to the additional link 165, along the horizontal plane.

Referring again to FIG. 12, a plane defining the angles to be changed by the angle change mechanism 122, that is, a plane including the arrow B1 or B2 is referred to as a plane P2. The plane P2 intersects with the plane P1.

The plane P1 is kept along the horizontal plane during the rotation of the third arm portion 137, as mentioned above. Thus, the angles, defined by the plane P1, of the angle change mechanism 122, which has the first arm portion 135, and of the base 121 and the gripping portion 124, which are connected to the angle change mechanism 122, are maintained.

That is, the third arm portion 137 rotates while maintaining the angles of the base 121 and the gripping portion 124 as viewed along the direction of the arrow V1, along which the two parallel link members 161 and 162 are arranged next to each other.

The rotation of the third arm portion 137 changes the heights of the base 121 and the gripping portion 124 as described above, and also changes the angles of the base 121 and the gripping portion 124 which are defined by the plane P2.

That is, the angle change mechanism 122 changes the angles of the base 121 and the gripping portion 124 as viewed along the direction of the arrow V2, along which the two first coupling portions 134 are arranged next to each other.

### Variations

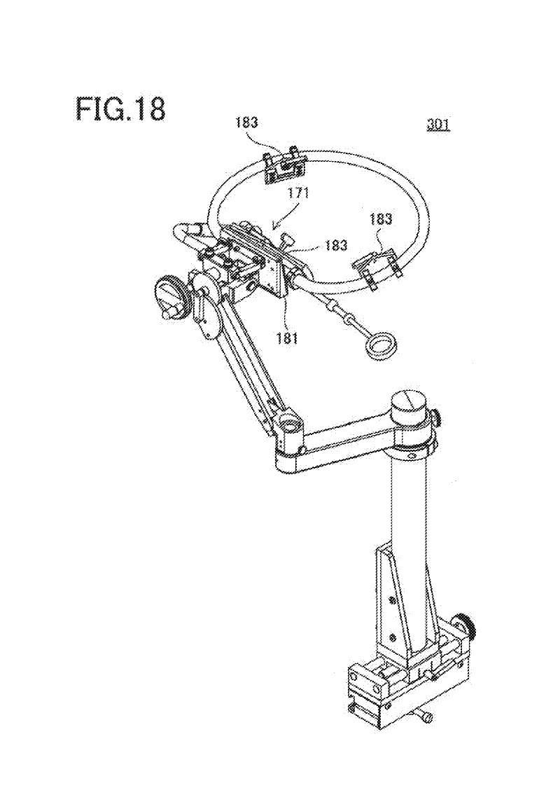

FIG. 18 is a diagram illustrating a configuration of a variation of the grip mechanism according to one or more embodiments.

Referring to FIG. 18, the grip mechanism 301 may include a base 171 in place of the base 121 illustrated in FIG. 10.

Specifically, the base 171 has a first support portion 181, a frame 182, and one or more attachment portions 183. The frame 182 is attached to the first support portion 181 and forms a circular loop, for example. The attachment portion 183 is disposed at any position of the frame 182. The surgical instrument 1 illustrated in FIG. 2 is attachable to the attachment portion 183.

The frame 182 may form, for example, a rectangular loop instead of a circular loop.

In one or more embodiments, the grip mechanism configured to grip the medical treatment tool 101 which utilizes the surgical instrument 1, guide tube 11, and bundling tube 12, each having a flexible portion, has been described as an example. Needless to say, the grip mechanism may be applicable not only to the medical treatment tool 101 which utilizes the surgical instrument 1, guide tube 11, and bundling tube 12, each having a flexible portion, but also to any mechanisms configured to activate a wide range of medical treatment tools.

For example, in the case of using a hard shaft instead of the flexible shaft 2 for the surgical instrument 1, the gripping portion 124 may be omitted from the grip mechanism of one or more embodiments. The grip mechanism of one or more embodiments may also be applicable to a laparoscopic surgery or any other surgeries involving a plurality of holes formed in the body surface without using the guide tube 11 and/or the bundling tube 12.

One or more embodiments disclosed herein are meant to be illustrative in all respects and should not be construed to be limiting in any manner. The scope of one or more embodiments is defined not by the above description, but by the scope of claims, and intended to include all modifications within equivalent meaning and scope to those of the claims.

The described one or more embodiments may be summarized as follows.

[1] A grip mechanism to grip a medical treatment tool, the grip mechanism comprising:
a base to which the medical treatment tool is attachable;
a vertical movement mechanism configured to change a height of the base; and
an angle change mechanism configured to change an angle of the base in conjunction with the vertical movement mechanism.

[2] The grip mechanism of [1] further comprising
an operating portion, wherein
the angle change mechanism and the vertical movement mechanism convert a force applied by the operating portion to a force that changes the angle of the base and a force that changes the height of the base, respectively.

[3] The grip mechanism of [1] or [2], wherein
the base includes a first support portion coupled to the angle change mechanism, and
the angle change mechanism changes an angle of the base such that a position of the first support portion of the base is lowered as the vertical movement mechanism lowers a position of the base.

[4] The grip mechanism of [3], wherein
the angle change mechanism changes the angle of the base by rotating the first support portion on an end of the first support portion in a vertical direction.

[5] The grip mechanism of [3] or [4], wherein
the angle change mechanism includes:
a first coupling portion; and
a second coupling portion which intersects with the first coupling portion as viewed along the vertical direction,
the first support portion has a first end in the vertical direction coupled to the first coupling portion, and a second end in the vertical direction coupled to the second coupling portion, and
the first coupling portion moves along a longitudinal direction of its own, allowing the first support portion to rotate about the second coupling portion, thereby changing the angle of the base.

[6] The grip mechanism of any one of [1] to [5], wherein
the vertical movement mechanism includes
an arm portion, and
the base is arranged at a first end of the arm portion via the angle change mechanism, and
the height of the base is changed by rotation of the arm portion on a second end of the arm portion.

[7] The grip mechanism of [6], wherein
the vertical movement mechanism further includes
a first gear arranged toward the first end of the arm portion and fixed with respect to the arm portion, and
a second gear which engages with the first gear,
planes of rotation of the first gear and the second gear are parallel to a plane of rotation of the arm portion, and
rotation of the second gear causes the first gear and the arm portion to rotate on the second end of the arm portion.

[8] The grip mechanism of [6] or [7], wherein
the vertical movement mechanism further includes
two link members extending along a longitudinal direction of the arm portion and parallel to each other, and
a joint member coupling the two link members, and
the joint member is connected to the base via the angle change mechanism, and rotates on the second end of the arm portion, in conjunction with the rotation of the arm portion, while maintaining an angle defined by a plane different from a plane defining the angle to be changed by the angle change mechanism.

[9] The grip mechanism of any one of [1] to [8], wherein
the medical treatment tool includes
a surgical instrument, and
an insertion tube in which the surgical instrument is to be inserted, and
the grip mechanism further comprises an insertion tube gripping portion gripping the insertion tube.

[10] The grip mechanism of [9], wherein
the insertion tube gripping portion includes a second support portion coupled to the angle change mechanism,
the vertical movement mechanism is further configured to change a height of the insertion tube gripping portion, and
the angle change mechanism changes an angle of the insertion tube gripping portion such that a position of the second support portion of the insertion tube gripping portion is lowered as the vertical movement mechanism lowers a position of the insertion tube gripping portion.

[11] The grip mechanism of any one of [1] to [10], wherein
the base has a circumferentially extending shape, part of which is open.

[12] The grip mechanism of any one of [1] to [10], wherein
the base forms a rectangular or circular loop.

[13] The grip mechanism of any one of [1] to [12] further comprising
a support column extending in a vertical direction, wherein
the base, the vertical movement mechanism, and the angle change mechanism are rotatable about the support column.

1. A surgical system, comprising:
a first surgical instrument including an elongate flexible first shaft, a first end effector arranged at a distal end portion of the first shaft, and a first activation mechanism arranged at a proximal end portion of the first shaft and configured to activate the first end effector; and
a second surgical instrument including an elongate flexible second shaft, a second end effector arranged at a distal end portion of the second shaft, and a second activation mechanism arranged at a proximal end portion of the second shaft and configured to activate the second end effector;
a bundling tube which is capable of being inserted in a patient and in which the first shaft and the second shaft are inserted;
a gripping portion configured to grip the bundling tube;
a base including a frame, a first attachment portion supported by the frame and configured to attach a part of the first surgical instrument and a second attachment portion supported by the frame and configured to attach a part of the second surgical instrument;
a vertical movement mechanism configured to change a height of the base; and
an angle change mechanism configured to change an angle of the base in conjunction with the vertical movement mechanism.

2. The surgical system of claim 1, further comprising:
an operating portion coupled to the angle change mechanism and the vertical movement mechanism, wherein
the angle change mechanism and the vertical movement mechanism are configured to convert a force applied by the operating portion to a force that changes the angle of the base and a force that changes the height of the base, respectively.

3. The surgical system of claim 2, wherein
the base has a rotational axis thereof parallel to a horizontal plane, and
the angle change mechanism includes a mechanism that converts the force applied by the operating portion to a force orthogonal to the rotational axis.

4. The surgical system of claim 3, wherein
the mechanism that converts the force applied by the operating portion to the force orthogonal to the rotational axis includes a ball screw and a nut portion.

5. The surgical system of claim 1, wherein
the angle change mechanism changes the angle of the base such that an angle between a longitudinal direction of the first surgical instrument and a horizontal direction becomes smaller as a height of the base is lowered by the vertical movement mechanism.

6. The surgical system of claim 1, wherein
the vertical movement mechanism includes an arm portion including a first end and a second end,
the base is arranged at the first end of the arm portion via the angle change mechanism, and
the height of the base is changed by rotation of the arm portion on the second end.

7. The surgical system of claim 6, wherein
the vertical movement mechanism further includes
two link members extending along a longitudinal direction of the arm portion and parallel to each other, and
a link coupling member coupling the two parallel link members, and
the link coupling member is connected to the base via the angle change mechanism, and rotates on the first end of the arm portion, in conjunction with the rotation of the arm portion, while maintaining an angle defined by a plane different from a plane defining the angle to be changed by the angle change mechanism.

8. The surgical system of claim 1, wherein
the base is configured such that proximal ends of the first and second surgical instruments are attachable to the base, and
the angle change mechanism is configured to change the angle of the base and an angle of the gripping portion.

9. The surgical system of claim 1, wherein
the angle change mechanism changes an angle between a longitudinal direction of the first surgical instrument and a horizontal direction by changing the angle of the base.

10. The surgical system of claim 1, wherein
the bundling tube includes a first guide tube through which the first shaft of the first surgical instrument extends, and a second guide tube through which the second shaft of the second surgical instrument extends.

11. The surgical system of claim 10, wherein
the bundling tube has a guide portion which guides insertion of the first and second guide tubes.

12. The surgical system of claim 1, further comprising a third surgical instrument, wherein
the third surgical instrument includes an endoscope including an elongate flexible third shaft and a camera arranged at a distal end of the third shaft,
the base is configured such that a part of the endoscope is attachable to the base, and
the bundling tube is configured such that the third shaft is inserted in the bundling tube.

13. The surgical system of claim 1, wherein
the frame has a circumferentially extending shape, part of which is open.

14. The surgical system of claim 1, wherein
the frame forms a rectangular or circular loop.

15. The surgical system of claim 1, further comprising:
a support column extending in a vertical direction, wherein
the base, the vertical movement mechanism, and the angle change mechanism are rotatable about the support column.

16. The surgical system of claim 1, further comprising:
a horizontal position adjusting mechanism configured to change a position of the base on a plane parallel to a horizontal plane.

17. A surgical system, comprising:
a first surgical instrument including an elongate flexible first shaft, a first end effector arranged at a distal end portion of the first shaft, and a first activation mechanism arranged at a proximal end portion of the first shaft and configured to activate the first end effector; and
a second surgical instrument including an elongate flexible second shaft, a second end effector arranged at a distal end portion of the second shaft, and a second activation mechanism arranged at a proximal end portion of the second shaft and configured to activate the second end effector;
a bundling tube including a first end portion and a second end portion, holding therein the first and second shafts of the first and second surgical instruments inserted from the first end portion, such that the first and second end effectors can be inserted from the second end portion into a patient's body;
a gripping portion configured to grip the bundling tube;
a base including a frame, a first attachment portion supported by the frame and configured to attach a part of the first surgical instrument and a second attachment portion supported by the frame and configured to attach a part of the second surgical instrument;
a vertical movement mechanism configured to change a height of the base; and
an angle change mechanism configured to change an angle of the base in conjunction with the vertical movement mechanism.

18. The surgical system of claim 17, wherein
the base is configured such that proximal ends of the first and second surgical instruments are attachable to the base, and
the angle change mechanism is configured to change the angle of the base and an angle of the gripping portion.

19. The surgical system of claim 17, wherein
the bundling tube includes a first guide tube through which the first shaft of the first surgical instrument extend, and a second guide tube through which the second shaft of the second surgical instrument extends.

20. A surgical system, comprising:
a surgical instrument including an elongate flexible first shaft, an end effector arranged at a distal end portion of the first shaft, and a first activation mechanism arranged at a proximal end portion of the first shaft and configured to activate the end effector;
an endoscope including an elongate flexible second shaft, a camera arranged at a distal end of the second shaft, and a second activation mechanism arranged at a proximal end portion of the second shaft and configured to activate the camera;
a bundling tube including a first end portion and a second end portion, holding therein the first shaft of the surgical instrument and the second shaft of the endoscope inserted from the first end portion, such that the end effector and the camera can be inserted from the second end portion into a patient's body;
a gripping portion configured to grip the bundling tube;
a base including a frame, a first attachment part supported by the frame and configured to attach a part of the surgical instrument and a second attachment part supported by the frame and configured to attach a part of the endoscope;
a vertical movement mechanism configured to change a height of the base; and
an angle change mechanism configured to change an angle of the base in conjunction with the vertical movement mechanism.
